(19) United States
(12) Patent Application Publication
(10) Pub. No.: US 2018/0228361 A1
(43) Pub. Date: Aug. 16, 2018

(54) ARTHROSCOPIC SYSTEM WITH DISPOSABLE ARTHROSCOPE

(71) Applicant: DYNACOLOR, INC., Taipei (TW)

(72) Inventors: Chung-Sheng Chen, Taipei (TW); Ching-Chuan Jiang, Taipei (TW); Chien-Hsiang Chang, Taipei (TW); Wei-lin Chen, Taipei (TW)

(21) Appl. No.: 15/432,943

(22) Filed: Feb. 15, 2017

(51) Int. Cl. A61B 1/317; A61B 1/00; A61B 1/05; A61B 1/06

(52) U.S. Cl. CPC A61B 1/317; A61B 1/00103; A61B 1/05; A61B 1/00114; A61B 1/00009; A61B 1/00045; A61B 1/00018; A61B 1/0676

(57) ABSTRACT

The present application provides an arthroscopic system, which comprises an arthroscope having a distal end and a proximal end, a light source attached to the distal end of the arthroscope for providing a light, a lens attached to the distal end of the arthroscope for gathering the light, an image sensor set up next to the lens for generating digital raw data from the light, a transmitter set up between the distal end and the proximal end for transmitting the digital raw data toward the proximal end, a processing system attached to the proximal end for capturing and converting the digital raw data into digital video data and a video display device for displaying said digital video data. The present application eliminates the possibility of infecting the patient with contaminated devices by its cost-effectively disposable arthroscope. The present application also provides a method for image transmitting in an arthroscopic system.

### FIELD OF THE INVENTION

The present application generally relates to an arthroscopic system in which image is captured and transmitted from the lens to video display devices. More particularly, the present application relates to the arthroscopic system with the disposable arthroscope.

### BACKGROUND OF THE INVENTION

Arthroscopy is a minimally invasive surgical procedure used to diagnose and treat a wide range of joint problems such as meniscal tears, and cartilage injury, etc. Arthroscopy allows doctors to view the joint without making a large incision through the skin and other soft tissues.

A typical arthroscopic system includes a camera module. The camera module includes an image sensor, digital signal processor (DSP) and a transmitter. To display an image for doctors to operate surgery, the image sensor captures the light which is generated by a light source and reflected by the object, and converts it into digital raw data. The digital raw data is then converted into digital video data in RGB/YUV format by a digital signal processor in the camera module in serial digital interface (SDI) standard. The transmitter in the camera module then transmits the digital video data to a video captured device and then the video data is displayed on video display device for the doctor to operate surgery for patient.

Arthroscopic examination typically involves inserting an arthroscope into the patient's joint to be examined. Existing arthroscopes are typically reusable devices and must accordingly be sterilized before each use to eliminate the possibility of infecting the patient with contaminated devices. Nevertheless the possibility of infection remains if the arthroscope is not effectively or completely sterilized. Furthermore, the arthroscope may become septic in the time period between sterilization and use of the arthroscope.

Disposal of the arthroscope is an option to avoid such infection; however it significantly raises the cost on operating arthroscopy because the digital signal processor (DSP) in the camera module is costly. Thus, it is an object of the present application to provide an arthroscopic system with disposable arthroscope which is relatively cost-effective and decrease the risk of infection in arthroscopic surgery.

### SUMMARY OF THE INVENTION

In one aspect, the present application relates to an arthroscopic system having

an arthroscope having a distal end and a proximal end, a light source attached to the distal end of the arthroscope for providing a light, a lens attached to the distal end of the arthroscope for gathering the light, an image sensor set up next to the lens for generating digital raw data from the light, a transmitter set up between the distal end and the proximal end for transmitting the digital raw data toward the proximal end, a processing system attached to the proximal end for capturing and converting the digital raw data into digital video data, and a video display device for displaying the digital video data.
In an embodiment, the processing system is a video capture and image processing system.
Preferably, the arthroscope is disposable.
In an embodiment, the arthroscope further comprises a cable.
Preferably, the transmitter is a Serializer/Deserializer (SerDes). In another embodiment, the transmitter is a low-voltage differential signaling device.
Preferably, the processing system converts digital raw data into digital video data in RGB/YUV format to be displayed by the video display device. In an embodiment the processing system has a digital signal processor. In another embodiment, the processing system has a computer with a central processing unit. In yet another embodiment, the processing system has a computer with a graphic card.
In another aspect, the present application provides a method for image transmitting in an arthroscopic system. The method includes a light generating step, where a light is generated by a light source; a light collecting step, wherein the light generated by the light source is collected by a lens; a converting step, wherein said light collected by said lens is converted into digital raw data by an image sensor; a transmitting step, wherein the digital raw data is transmitted from a distal end of an arthroscope to a proximal end of the arthroscope by at least a transmitter; a video capture and image processing step, wherein the digital raw data transmitted by said transmitter is captured and processed into digital video data; and a displaying step, wherein the digital video data is displayed by a video display device.
Preferably, the transmitter is a Serializer/Deserializer (SerDes). In another embodiment, the transmitter is a low-voltage differential signaling device.
Preferably, the method has a use step wherein the distal end of the arthroscope contacts with a patient.
Preferably, the method has a disposal step wherein said arthroscope comprising light source, lens, image sensor and transmitter is thrown away.

### BRIEF DESCRIPTION OF THE DRAWINGS

This application can be better understood with reference to the following drawings and description.

FIG. 1 presents a module chart of a typical arthroscopic system.

FIG. 2 presents a module chart of another typical and improved arthroscopic system.

FIG. 3 presents a module chart of a novel disposable arthroscope of the present application.

FIG. 4 presents a process flow diagram of a method for image transmitting in an arthroscopic system.

### DETAILED DESCRIPTION OF THE INVENTION

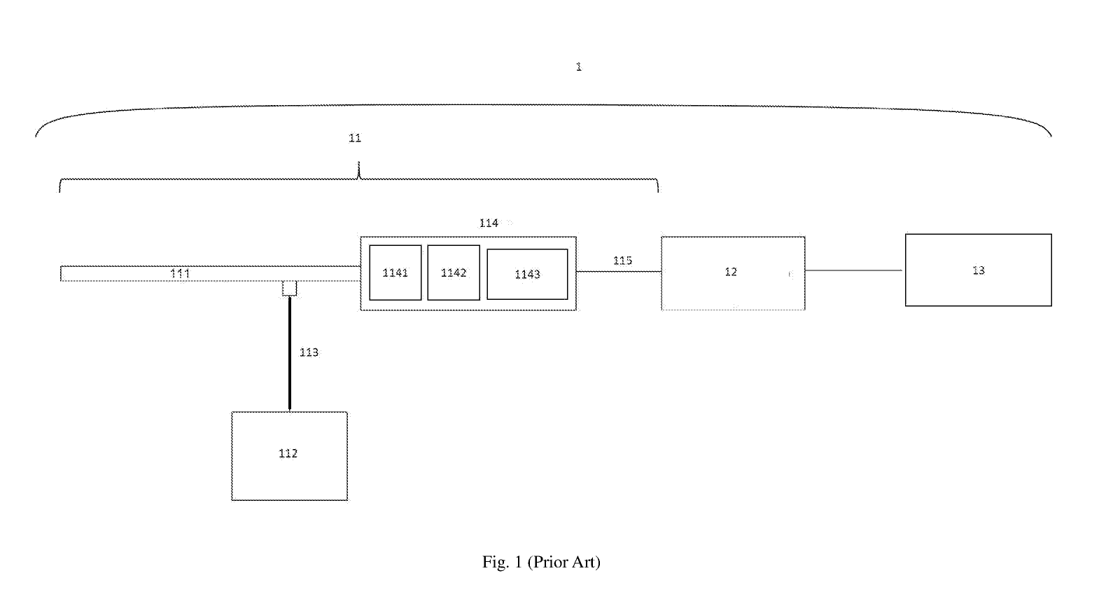

Referring initially to FIG. 1, a typical arthroscopic system 1 includes an arthroscope 11, a video capture device 12 and a video display device 13. The arthroscope 11 has the lens set 111, the light source 112, light cord 113, and the camera module 114. The camera module 114 has the image sensor 1141, the digital signal processor (DSP) 1142 and the transmitter 1143. Between the transmitter 1143 and the video capture device 12 there is a cable 115.

The disadvantage of the system is that the light source 112 is large and bulky in order to generate high-intensity light for the system 1 and the arthroscope 11. Along with the costly camera module 114, the arthroscope 11 in the arthroscopic system 1 as shown in FIG. 1 is too expensive to be disposable and requires sterilization process after used, causing the concern of infection.

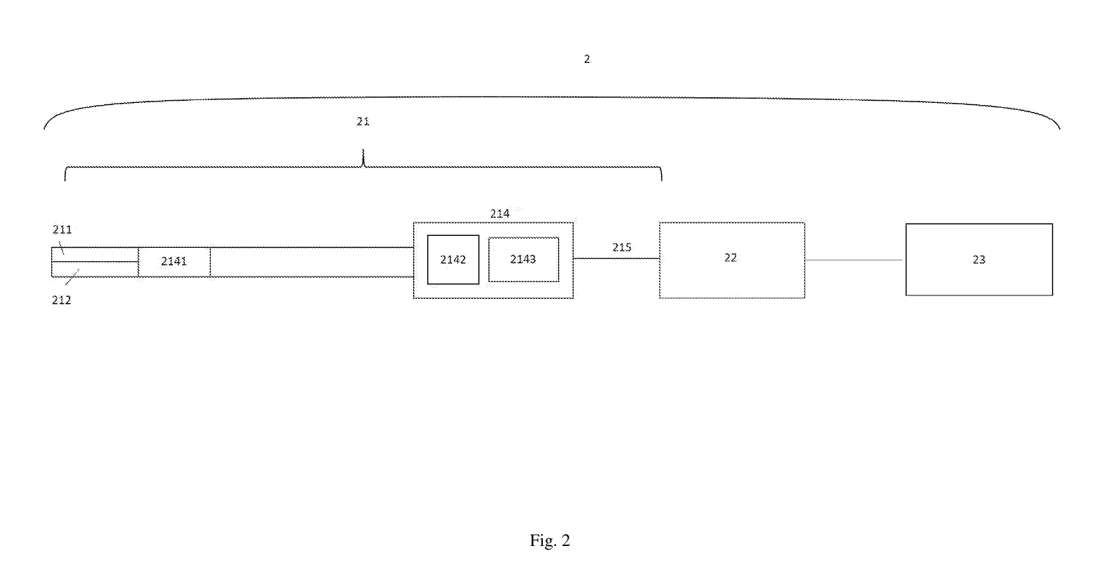

FIG. 2 shows a module chart of an improved arthroscopic system 2 having the arthroscope 21, the video capture device 22, and the video display device 23. The light source 212, the lens set 211 and the image sensor 2141 are placed to the tip of distal end of the arthroscope 21 to eliminate the need of a bulky light source. The cable 215 is placed between the camera module 214 and the video capture device 22. The camera module 214 includes the DSP 2142 and transmitter 2143. However the arthroscopic system 2 still requires an expensive camera module 214 in the arthroscope 21 and hence disposal of such arthroscope 21 is unlikely.

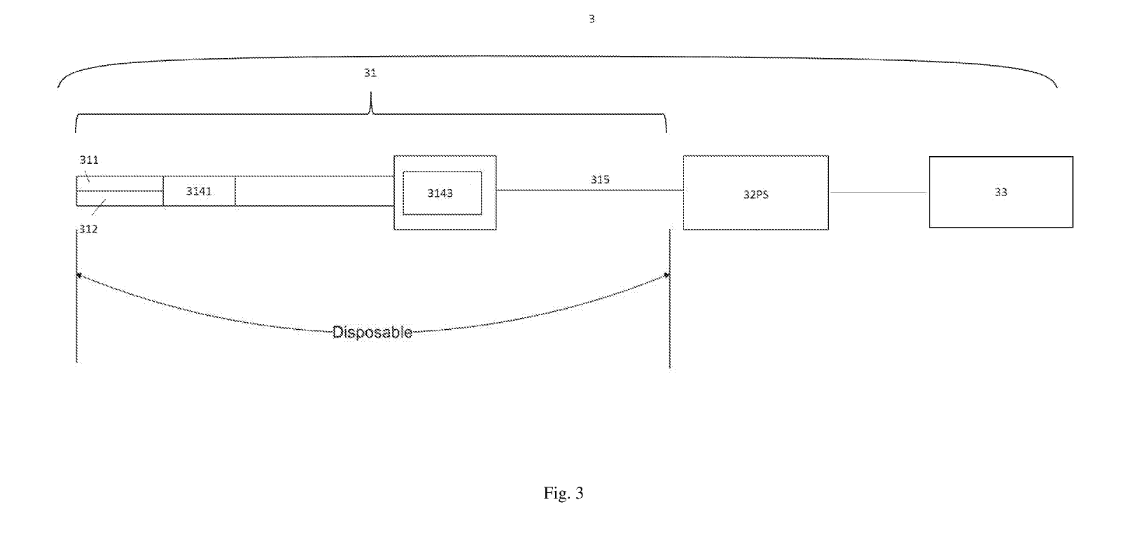

FIG. 3 presents a module chart of an arthroscopic system 3 with disposable arthroscope 31 of the present application. Comparing to the typical arthroscopic system 1 as shown in FIG. 1, the present arthroscopic system 3 places the light source 312, lens set 311 and image sensor 3141 to the distal end of the arthroscope 31 to eliminate the need of a bulky light source 112 as shown in FIG. 1. The light source 312 of the present arthroscopic system includes but not limited to light-emitting diode (LED) and laser diode (LD), and the image sensor 3141 includes but not limited to a Complementary Metal-Oxide-Semiconductor (CMOS).

Digital raw data generated by the image sensor 3141 is directly transmitted to the processing system 32PS which is attached to the proximal end of the arthroscope 31 via the transmitter 3143 and a cable 315.

In this embodiment as shown in FIG. 3, the processing system 32PS is in charge of video capturing and image processing and is composed of known components in the field to perform its features. The transmitter 3143 includes but not limited to Serializer/Deserializer (SerDes) and low-voltage differential signaling (LVDS) device. Digital raw data is captured by the processing system 32PS to preserve raw data for future use and convert digital raw data into digital video data in RGB/YUV format to be displayed by the video display device 33. The video display device 33 includes but not limited to a screen. In the present arthroscopic system 3, there is no DSP in arthroscope 31, therefore the arthroscope 31 can be thrown away in a cost-effectively way.

Referring back to FIG. 1 and FIG. 3, by removing the image processing feature performed by DSP 1142 from the arthroscope 11, and by applying transmitter 3143 which can transmit the digital raw data from the distal end to the processing system 32PS attached to the proximal end of the arthroscope 31 without a costly DSP 1142, the present application makes the arthroscope 31 cost-effectively disposable and significantly decreases the risk of infection.

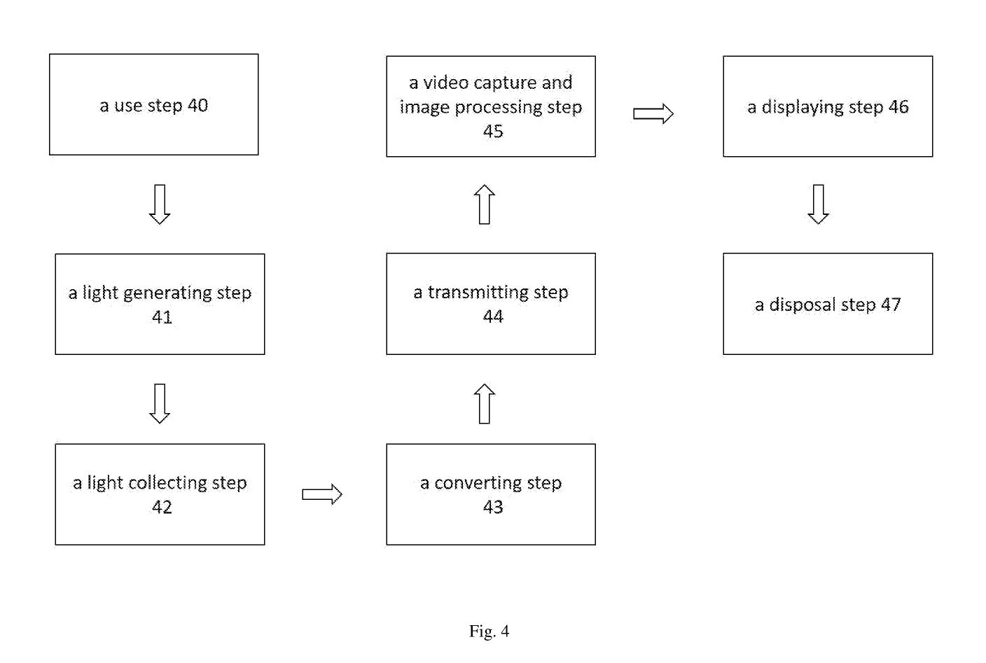

Referring to FIG. 3 and FIG. 4, to apply the present application, there is a use step 40 wherein the arthroscope 31 contacts a patient to diagnose or to perform a surgery. The light source 312 conducts a light generating step 41. The generated light is collected by lens sets 311 in a light collecting step 42. The collected light is converted into digital raw data in a converting step 43 by an image sensor 3141, and in a transmitting step 44 the digital raw data is transmitted from a distal end of an arthroscope 31 to a proximal end of the arthroscope 31 by at least a transmitter 3143 and a cable 315. The transmitter 3143 is preferably a Serializer/Deserializer (SerDes) or a low-voltage differential signaling device. In a video capture and image processing step 45, the digital raw data is captured and processed into digital video data by the processing system 32PS. In a displaying step 46, the digital video data is displayed on a video display device 33. After the diagnose or the surgery, the arthroscope 31 which contacts the patient is thrown away with the lens sets 311, light source 312, image sensor 3141, transmitter 3143, and cable 315 to avoid infection to the next patient. While most of the typical arthroscopic systems apply SDI standard for transmitting data in RGB/YUV format through the arthroscope to the video capture device, the present application applies transmitter 3143 such as SerDes and LVDS device to extend the distance for transmitting digital raw data, and moves the image processing feature to the processing system 32PS. In an embodiment, the processing system 32PS conducts the image processing feature by a DSP component in the processing system 32PS. In some other embodiments, the processing system 32PS conducts the image processing feature by a central processing unit or a graphic card in a computer. Hence, without having an expensive DSP in the arthroscope which contacts with patient during a surgery, the arthroscope in the arthroscopic system is cost-effectively disposable.

What is claimed is:

1. An arthroscopic system comprising:
an arthroscope having a distal end and a proximal end;
a light source attached to said distal end of said arthroscope for providing a light;
a lens attached to said distal end of said arthroscope for gathering said light;
an image sensor set up next to said lens for generating digital raw data from said light;
a transmitter set up between said distal end and said proximal end for transmitting said digital raw data toward said proximal end;
a processing system attached to said proximal end for capturing and converting said digital raw data into digital video data, and
a video display device for displaying said digital video data.

2. The arthroscopic system of claim 1, wherein said processing system is a video capture and image processing system.

3. The arthroscopic system of claim 1, wherein said arthroscope is disposable.

4. The arthroscopic system of claim 3, wherein said arthroscope further comprises a cable.

5. The arthroscopic system of claim 3, wherein said transmitter is a Serializer/Deserializer (SerDes).

6. The arthroscopic system of claim 3, wherein said transmitter is a low-voltage differential signaling device.

7. The arthroscopic system of claim 3, wherein said processing system converts digital raw data into digital video data in RGB/YUV format to be displayed by said video display device.

8. The arthroscopic system of claim 7, wherein said processing system comprises a digital signal processor.

9. The arthroscopic system of claim 7, wherein said processing system comprises a computer with a central processing unit.

10. The arthroscopic system of claim 7, wherein said processing system comprises a computer with a graphic card.

11. A method for image transmitting in an arthroscopic system, comprising:
a light generating step, wherein a light is generated by a light source,
a light collecting step, wherein said light generated by said light source is collected by a lens;
a converting step, wherein said light collected by said lens is converted into digital raw data by an image sensor;
a transmitting step, wherein said digital raw data is transmitted from a distal end of an arthroscope to a proximal end of said arthroscope by at least a transmitter;
a video capture and image processing step, wherein the digital raw data transmitted by said transmitter is captured and processed into digital video data; and
a displaying step, wherein said digital video data is displayed by a video display device.

12. The method for image transmitting in an arthroscopic system as claimed in claim 11, wherein said transmitter is a Serializer/Deserializer (SerDes).

13. The method for image transmitting in an arthroscopic system as claimed in claim 11, wherein said transmitter is a low-voltage differential signaling device.

14. The method for image transmitting in an arthroscopic system as claimed in claim 12 furthering comprising a use step wherein said distal end of said arthroscope contacts with a patient.

15. The method for image transmitting in an arthroscopic system as claimed in claim 12 furthering comprising a disposal step wherein said arthroscope comprising light source, lens, image sensor and transmitter is thrown away.
